(19) United States
(12) Patent Application Publication
(10) Pub. No.: US 2021/0244903 A1
(43) Pub. Date: Aug. 12, 2021

(54) INTRODUCER/EXCHANGER FOR TRACHEOSTOMY TUBE

(71) Applicant: UNITY HEALTH TORONTO, TORONTO (CA)

(72) Inventor: João Baptista De Rezende Neto, TORONTO (CA)

(73) Assignee: UNITY HEALTH TORONTO, TORONTO, ON (CA)

(21) Appl. No.: 17/269,299

(22) PCT Filed: Sep. 19, 2019
(86) PCT No.: PCT/CA2019/051328

§ 371 Date: Feb. 18, 2021

Related U.S. Application Data
(60) Provisional application No. 62/737,039, filed on Sep. 26, 2018.

(51) Int. Cl. A61M 16/04

(52) U.S. Cl. CPC A61M 16/0488; A61M 2205/8206; A61M 2205/587; A61M 16/0472

(57) ABSTRACT

An introducer/exchanger for a tracheostomy device includes a flexible elongate member having a proximal end, a rounded blunt distal end, and a lumen extending from a proximal opening at the proximal end to a distal opening at the distal end. A distally pointing light is mounted to the rounded blunt distal end. A power source is provided for the light.

### CROSS-REFERENCES TO RELATED APPLICATIONS

This application claims the benefit of and/or priority from U.S. Provisional Patent Application No. 62/737,039, filed on Sep. 26, 2018, which is incorporated herein by reference in its entirety.

### FIELD

This document relates to tracheostomy procedures. More specifically, this document relates to introducing and exchanging devices for tracheostomy tubes and to methods for introducing a tracheostomy tube into a tracheostomy and/or exchanging a tracheostomy tube.

### BACKGROUND

US Patent Application Publication No. US20090163942A1 (Cuevas et al.) discloses a device for performing an initial piercing and dilating of a patient's trachea. The device has a needle within and extending beyond a sheath. After the needle is used to pierce the trachea, it may be removed and a guide wire (J-wire) inserted. An introducer dilator surrounds the sheath and is slidable over the sheath. The introducer dilator has a first position away from the distal end of the sheath and a second position in which the distal end of the sheath is substantially covered by the introducer dilator. The introducer dilator is then used to expand the initial piercing.

### SUMMARY

The following summary is intended to introduce the reader to various aspects of the detailed description, but not to define or delimit any invention.

According to some aspects, an introducer/exchanger for a tracheostomy tube includes a flexible elongate member having a proximal end, a rounded blunt distal end, and a lumen extending from a proximal opening at the proximal end to a distal opening at the distal end. A distally pointing light is mounted to the distal end. A power source is provided for the light.

In some examples, the light is an LED. The light can be annular and can extend around the distal opening.

In some examples, the flexible elongate member is a spiral wound wire or a spiral cut tube. The power source can include a battery mounted to the proximal end, and the battery can be connected to the light via a connector wound spirally along the flexible elongate member. The connector can be wound around an outside surface of the flexible elongate member, or wound inside the lumen of the flexible elongate member.

In some examples, the flexible elongate member is a plastic tube.

In some examples, the introducer/exchanger can be used to facilitate the insertion of a tracheostomy tube into a tracheostomy.

In some examples, a method for introducing and/or exchanging a tracheostomy tube into a tracheostomy includes a. approaching a tracheostomy with a rounded blunt distal end of an introducer/exchanger and Illuminating the tracheostomy with a light mounted to the distal end; b. passing the distal end of the introducer/exchanger into the tracheostomy; c. positioning a tracheostomy tube over the introducer/exchanger; d. sliding the tracheostomy tube into the tracheostomy; and f. removing the introducer/exchanger from the tracheostomy.

In some examples, step d. is carried out prior to step a.

In some examples, the method further includes, prior to step a., sliding the introducer/exchanger over a guidewire that is in the tracheostomy.

### BRIEF DESCRIPTION OF THE DRAWINGS

The drawings included herewith are for illustrating various examples of articles, methods, and apparatuses of the present specification and are not intended to limit the scope of what is taught in any way. In the drawings:

FIG. 1A is a perspective view of an example introducer/exchanger;

FIG. 1B is an enlarged view of a portion of the introducer/exchanger of FIG. 1A;

FIG. 2 is s a perspective view of the introducer/exchanger of FIG. 1, flexed to a bent configuration;

FIG. 3 is a perspective view of the distal end of the introducer/exchanger of FIG. 1;

FIG. 4 is an end view of the distal end of the introducer/exchanger of FIG. 1; and

FIG. 5 is a perspective view of the proximal end of the introducer/exchanger of FIG. 1.

### DETAILED DESCRIPTION

Various apparatuses or processes will be described below to provide an example of an embodiment of the claimed subject matter. No embodiment described below limits any claim and any claim may cover processes or apparatuses that differ from those described below. The claims are not limited to apparatuses or processes having all of the features of any one apparatus or process described below or to features common to multiple or all of the apparatuses described below. It is possible that an apparatus or process described below is not an embodiment of any exclusive right granted by issuance of this patent application. Any subject matter described below and for which an exclusive right is not granted by issuance of this patent application may be the subject matter of another protective instrument, for example, a continuing patent application, and the applicants, inventors or owners do not intend to abandon, disclaim or dedicate to the public any such subject matter by its disclosure in this document.

As used herein, the term “tracheostomy” refers to a surgically created hole through the trachea. The term “tracheotomy” refers to a surgical procedure that creates a tracheostomy. The term “tracheostomy tube” refers to a tube that is inserted into the trachea through an opening in the anterior aspect of the neck to maintain a patent airway.

Generally disclosed herein is a device for introducing and/or exchanging a tracheostomy tube. The device may also be referred to herein as an introducing/exchanging device or an introducer/exchanger. The introducer/exchanger has a light at its distal end, which can illuminate a tracheostomy. This can improve visibility during insertion of the introducer/exchanger and thereby facilitates insertion of the introducer/exchanger and the tracheostomy tube (e.g. in initial insertion or changing of a tracheostomy tube).

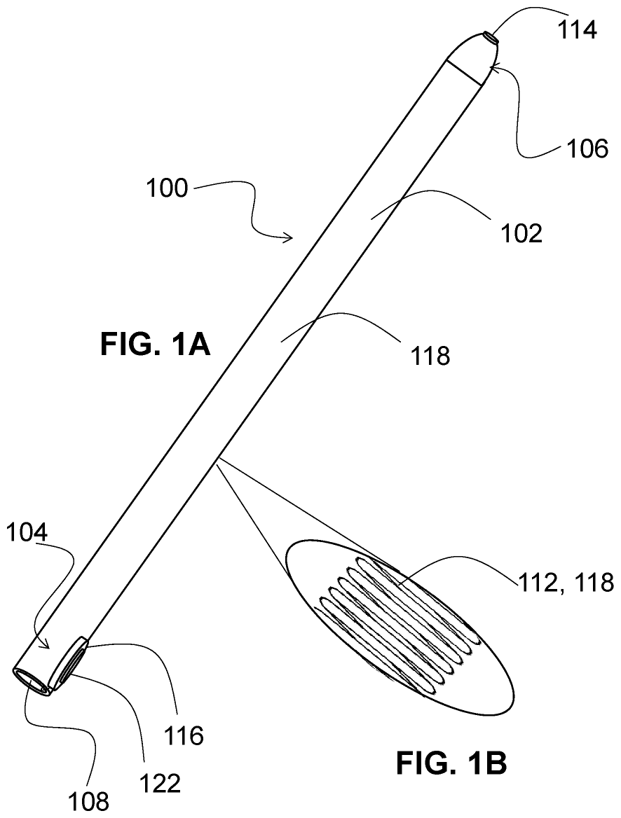

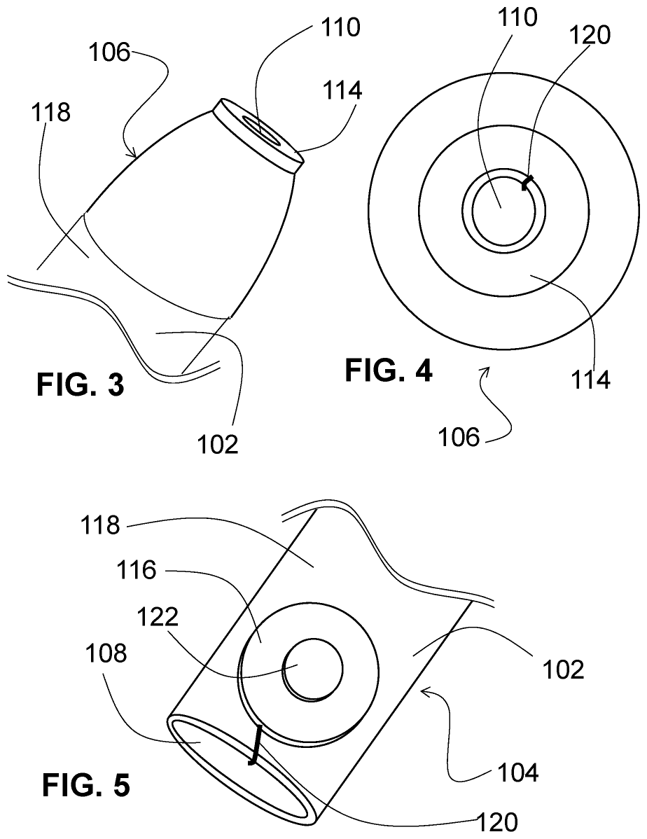

Referring now to FIG. 1A, an introducer/exchanger 100 for a tracheostomy tube is shown. The introducer/exchanger is an elongate member 102 having a proximal end 104 and a distal end 106. A lumen extends through the elongate member 102, from an opening at the proximal end 104 (referred to as a ‘proximal opening 108’) to an opening at the distal end 106 (referred to herein as a ‘distal opening 110’, seen in FIGS. 3 and 4). The distal end 106 is blunt and rounded.

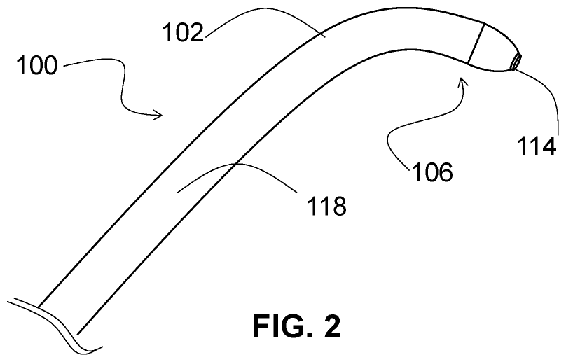

Referring to FIG. 2, the elongate member 102 is flexible, so that the distal end 106 can bend downwardly towards the lungs after being passed through a tracheostomy and into the trachea.

Referring back to FIG. 1 B, in the example shown, in order to provide flexibility, the elongate member 102 is fabricated from a spiral wound wire 112. For example, the elongate member 102 may be fabricated from a steel wire having a diameter of approximately 1 mm.

In alternative examples, the elongate member may be fabricated from a spiral cut tube, for example a spiral cut plastic tube. In yet further alternative examples, the elongate member may be a tube that is not spiral cut, and that is fabricated from a relatively flexible material.

Referring now to FIGS. 3 and 4, the introducer/exchanger 100 includes a light 114 for illuminating the tracheostomy during insertion of the introducer 100. More specifically, the light 114 is mounted to the distal end 106 and is distally pointing or shining. In the example shown, the light 114 is an annular shaped light emitting diode (LED), which is positioned around the distal opening 110.

In alternative examples, the light can be of another configuration. For example, the light can be a shape other than annular.

Referring to FIG. 5, the introducer/exchanger 100 further includes a power source for the light 114. In the example shown, the power source is a battery (not shown), which housed in a power unit 116 that is mounted to the proximal end 104, on an outside surface 118 of the elongate member 102. The power unit 116 further includes an on/off switch 122 for the light 114. In alternative examples, the power unit and the parts thereof can be mounted elsewhere (e.g. within the lumen, on an inside surface of the introducer), or can be spaced from the elongate member 102 (e.g. can be provided as a separate power unit).

The battery can be connected to the light 114 via an electrical connector such as a wire or cable. In the example shown, the electrical connector 120 extends from the power unit 116 to the light 114 via the lumen. In other examples, the electrical connector can run along the outside surface of the elongate member 102. In order to allow for the elongate member 102 to flex, the electrical connector 120 can have some slack to it, so that it can bend as the elongate member 102 flexes. Alternatively, the electrical connector 120 can be spiral wound (e.g. wound with the spiral wound wire that forms the elongate member 102), either on the outside surface 118 or the inside surface of the elongate member 102.

In some examples (not shown), a camera can also be provided at the distal end of the introducer/exchanger, proximate the light. The camera can be a video camera or a still-image camera. The camera can communicate images and/or videos to a display (e.g. a screen that is separate from the introducer/exchanger), to allow for visualization of the trachea.

In use, after a tracheostomy has been created in a trachea of a patient and a guidewire has been inserted through the tracheostomy, the light 114 of the introducer/exchanger 100 can be turned on, and the introducer/exchanger 100 can be slid over the guidewire towards the tracheostomy. As the distal end 106 of the elongate member 102 approaches the tracheostomy, the tracheostomy is illuminated by the light 114, allowing for better visibility of the tracheostomy. The distal end 106 of the elongate member 102 can then be passed into the tracheostomy, and optionally flexed downwardly over the guidewire. Due to the improved visibility provided by the light 114, passage of the distal end 106 of the elongate member 102 into the tracheostomy may be achieved with relative ease. A tracheostomy tube can then be positioned over the introducer/exchanger 100 and slid into the tracheostomy. The introducer/exchanger 100 can then be removed from the tracheostomy.

In an alternative example, the tracheostomy tube can be slid over the introducer/exchanger 100 prior to insertion of the introducer/exchanger 100 into the tracheostomy. In such examples, the tracheostomy tube can be slid over the introducer/exchanger 100 from the distal end 106 of the elongate member 102 towards the proximal end 104, or from the proximal end 104 of the elonage member 102 towards the distal end 106.

While the above description provides examples of one or more processes or apparatuses, it will be appreciated that other processes or apparatuses may be within the scope of the accompanying claims.

To the extent any amendments, characterizations, or other assertions previously made (in this or in any related patent applications or patents, including any parent, sibling, or child) with respect to any art, prior or otherwise, could be construed as a disclaimer of any subject matter supported by the present disclosure of this application, Applicant hereby rescinds and retracts such disclaimer. Applicant also respectfully submits that any prior art previously considered in any related patent applications or patents, including any parent, sibling, or child, may need to be re-visited.

1. An introducer/exchanger for a tracheostomy tube comprising:
a flexible elongate member having a proximal end, a blunt and rounded distal end, and a lumen extending from a proximal opening at the proximal end to a distal opening at the distal end;
a distally pointing light mounted to the distal end; and
a power source for the light.

2. The introducer/exchanger of claim 1, wherein light is an LED.

3. The introducer/exchanger of claim 1, wherein the light is annular and extends around the distal opening.

4. The introducer/exchanger of claim 1, wherein the flexible elongate member is a spiral wound wire or a spiral cut tube.

5. The introducer/exchanger of claim 1, wherein the power source comprises a battery mounted to the proximal end, and the battery is connected to the light via a connector wound spirally along the flexible elongate member.

6. The introducer/exchanger of claim 5, wherein the connector is wound around an outside surface of the flexible elongate member.

7. The introducer/exchanger of claim 5, wherein the connector is wound inside the lumen of the flexible elongate member.

8. The introducer/exchanger of claim 1, wherein the flexible elongate member is a plastic tube.

9. A use of the device of claim 1 to facilitate the insertion/exchange of a tracheostomy tube into a tracheostomy.

10. A method for introducing/exchanging a tracheostomy tube into a tracheostomy, comprising:
a. approaching a tracheostomy with a distal end of an introducer/exchanger and Illuminating the tracheostomy with a light mounted to the distal end;
b. passing the distal end of the introducer/exchanger into the tracheostomy;
c. positioning a tracheostomy tube over the introducer/exchanger;
d. sliding the tracheostomy tube into the tracheostomy; and
e. removing the introducer/exchanger from the tracheostomy.

11. The method of claim 10, wherein step c. is carried out prior to step a.

12. The method of claim 10, further comprising, prior to step a., sliding the introducer/exchanger over a guidewire that is in the tracheostomy.
